(19) United States
(12) Patent Application Publication
(10) Pub. No.: US 2023/0210053 A1
(43) Pub. Date: Jul. 6, 2023

(54) METHOD OF GRADIENT HARVESTING PLANT PRODUCT AND COMBINE HARVESTER FOR THE SAME

(71) Applicant: BENSON HILL, INC., St. Louis, MO (US)

(72) Inventors: Roger Keith O'Kelley, South Fulton, TN (US); Kevin McCowan, St. Louis, MO (US)

(73) Assignee: BENSON HILL, INC., St. Louis, MO (US)

(21) Appl. No.: 18/068,188

(22) Filed: Dec. 19, 2022

Related U.S. Application Data
(60) Provisional application No. 63/295,254, filed on Dec. 30, 2021.

(51) Int. Cl. A01D 41/14; A01D 41/127; A01D 45/22; G01N 21/3563; G01N 21/359; G01N 21/84; G01N 33/00; C07K 1/14; A23J 3/16; A23J 1/14; A23L 33/185

(52) U.S. Cl. CPC A01D 41/141; A01D 41/127; A01D 45/22; G01N 21/3563; G01N 21/359; G01N 21/84; G01N 33/0098; C07K 1/145; A23J 3/16; A23J 1/14; A23L 33/185; G01N 2021/8466

(57) ABSTRACT

A method of harvesting plant product from a plant in a single pass using a combine harvester is disclosed. In the method, the plant has a protein content gradient that varies along a height of the plant. The method includes identifying, along a longitudinally-extending stalk of the plant, an upper protein gradient of the plant including high protein plant product and a lower protein gradient of the plant including lower protein plant product, wherein the high protein plant product from the upper protein gradient of the plant meets a threshold protein content that is higher than that of the lower protein plant product. The method also includes separately and substantially simultaneously harvesting the high protein plant product from the upper protein gradient and the lower protein plant product from the lower protein gradient in the single pass, and isolating the high protein plant product from the lower protein plant product.

### TECHNICAL FIELD

The present disclosure relates generally to a method for harvesting a plant product, and more specifically to a method for gradient harvesting different portions of a soy plant having different protein contents in a single pass using a combine harvester.

### BACKGROUND

Soybeans represent an attractive renewable source of protein for use in foodstuffs. However, the protein content of unprocessed soybeans differs depending on which portion of the plant the soybeans are harvested from, i.e., soybeans harvested from a top portion of the plant are naturally higher in protein than soybeans harvested from a bottom portion of the plant closer to the ground.

Currently, soybeans are not gradient harvested based on protein content. Instead, soybeans are typically harvested such that the high and lower protein content soybeans are mixed together, or the gradients are independently harvested in separate passes by the combine harvester. The former method requires downstream fractionation in order to separate the high protein soybeans from the lower protein soybeans after harvest, which tends to be costly. The latter method is more time intensive and requires multiple passes by the combine harvester.

Thus, there is a need for a streamlined method of gradient harvesting the high and lower protein content soybeans simultaneously, such that the soybeans of differing protein contents remain separated from the point of harvest.

### BRIEF SUMMARY

The present disclosure relates to a method for gradient harvesting different portions of a soy plant in a single pass using a combine harvester. In some example embodiments, a method of gradient harvesting plant product from a plant in a single pass using a combine harvester, the plant having a protein content gradient that varies along a height of the plant, comprises: identifying, along a longitudinally-extending stalk of the plant, an upper protein gradient of the plant including high protein plant product and a lower protein gradient of the plant including lower protein plant product, wherein the high protein plant product in the upper protein gradient of the plant meets a threshold protein content that is higher than a protein content of the lower protein plant product in the lower protein gradient; separately and substantially simultaneously harvesting the high protein plant product from the upper protein gradient of the plant and the lower protein plant product from the lower protein gradient of the plant in the single pass of the combine harvester; and isolating the high protein plant product from the lower protein plant product.

In some example embodiments of the method of any previous or any combination of preceding example embodiments, identifying the upper protein gradient and the lower protein gradient comprises identifying a lower boundary of the high protein gradient, the lower boundary being defined by a height along the stalk of the plant of the plant product closest to the ground surface that has a protein content meeting the threshold protein content of the upper protein gradient.

In some example embodiments of the method of any previous or any combination of preceding example embodiments, the combine harvester comprises a first header including a first cutter bar and a second header including a second cutter bar, and wherein separately and substantially harvesting the high protein plant product from the upper protein gradient of the plant and the lower protein plant product from the lower protein gradient of the plant in the single pass comprises: making, using the first cutter bar, a first cut on the stalk of the plant proximate to the ground surface to detach the plant from the ground surface; and substantially simultaneously making, using the second cutter bar, a second cut on the stalk of the plant above the first cut and below the lower boundary to separate the upper protein gradient from the lower protein gradient.

In some example embodiments of the method of any previous or any combination of preceding example embodiments, the method further comprises separately analyzing, using a near-infrared spectroscopy (NIRS) mechanism in communication with the second header, a protein content of the high protein plant product harvested from the upper protein gradient of the plant and a protein content of the lower protein plant product harvested from the lower protein gradient of the plant.

In some example embodiments of the method of any previous or any combination of preceding example embodiments, separately and substantially harvesting the high protein plant product from the upper protein gradient of the plant and the lower protein plant product from the lower protein gradient of the plant further comprises: automatically adjusting a height of the first header relative to the ground surface; and automatically adjusting a height of the second header relative to the first header and based on real-time feedback from the NIRS mechanism on the protein content of the high protein plant product harvested from the upper protein gradient of the plant and the protein content of the lower protein plant product harvested from the lower protein gradient of the plant so as to automatically adjust a height of the second cut.

In some example embodiments of the method of any previous or any combination of preceding example embodiments, wherein automatically adjusting the height of the second header comprises: automatically adjusting the height of the second header closer to the height of the first header in response to the real-time feedback from the NIRS mechanism that the lower protein plant product harvested from the lower protein gradient of the plant includes a percentage of plant product with a protein content that meets the threshold protein content for the upper protein gradient, or automatically adjusting the height of the second header farther from the height of the first header in response to the real-time feedback from the NIRS mechanism that the higher protein plant product harvested from the upper protein gradient of the plant includes a percentage of plant product with a protein content that does not meet the threshold protein content for the upper protein gradient.

In some example embodiments of the method of any previous or any combination of preceding example embodiments, wherein the combine harvester comprises a separation mechanism, and wherein separately and substantially simultaneously harvesting the high protein plant product from the upper protein gradient of the plant and the lower protein plant product from the lower protein gradient of the plant comprises separately threshing and separating the lower protein plant product from the plant and threshing and separating the high protein plant product from the plant.

In some example embodiments of the method of any previous or any combination of preceding example embodiments, wherein the separation mechanism comprises a partition arranged to divide the separation mechanism into a first separator in communication with the first header and a second separator in communication with the second header, and wherein separately and substantially simultaneously harvesting the high protein plant product from the upper protein gradient of the plant and the lower protein plant product from the lower protein gradient of the plant comprises conveying, using a first conveyor, the lower protein plant product from the first header to the first separator to thresh and separate the lower protein plant product from the plant, and conveying, using a second conveyor, the high protein plant product from the second header to the second separator to thresh and separate the high protein plant product from the plant.

In some example embodiments of the method of any previous or any combination of preceding example embodiments, wherein the combine harvester comprises a storage tank, and wherein isolating the high protein plant product from the lower protein plant product comprises isolating the high protein plant product from the lower protein plant product in the storage tank.

In some example embodiments of the method of any previous or any combination of preceding example embodiments, wherein the storage tank comprises a partition arranged to divide the storage tank into a first storage tank and a second storage tank, and wherein isolating the high protein plant product from the lower protein plant product in the storage tank comprises storing the lower protein plant product in the first storage tank and storing the high protein plant product in the second storage tank.

In some example embodiments of the method of any previous or any combination of preceding example embodiments, the plant is a soy plant producing soybeans and the threshold protein content for the upper protein gradient of the soy plant is 40%, 42%, or 45% on a dry weight basis.

In some example embodiments of the method of any previous or any combination of preceding example embodiments, the high protein plant product is used to create a protein-enriched soy protein composition, the method comprising: subjecting the isolated high protein plant product to one of defatting by solvent extraction, desolventizing, and contacting with a polar solvent, to arrive at a protein-enriched soy protein composition that comprises at least about 75% soy protein on a dry weight basis.

In some example embodiments of the method of any previous or any combination of preceding example embodiments, the protein-enriched soy protein composition that comprises at east about 75% soy protein on a dry weight basis is a soy protein concentrate (SPC).

In some other example embodiments, a method of producing a food product, a beverage product, a dietary supplement product or other product, comprises the protein-enriched soy composition according to any previous or any combination of preceding example embodiments.

In some further example embodiments, a combine harvester for gradient harvesting plant product from a plant in a single pass, the plant having a protein content gradient that varies along a height of the plant, comprises: a first header including a first cutter bar for making a first cut on a stalk of the plant proximate to a ground surface to detach the plant from the ground surface; a second header including a second cutter bar for making a second cut on the stalk of the plant above the first cut to separate an upper protein gradient of the plant from a lower protein gradient of the plant, the upper protein gradient including high protein plant product and the lower protein gradient of the plant including lower protein plant product, wherein the high protein plant product in the upper protein gradient of the plant meets a threshold protein content that is higher than a protein content of the lower protein plant product in the lower protein gradient; a separation mechanism arranged to separately thresh and separate the lower protein plant product from the plant and thresh and separate the high protein plant product from the plant; and a storage tank arranged to isolate the high protein plant product from the lower protein plant product.

In some example embodiments of the combine harvester of any previous or any combination of preceding example embodiments, the second cutter bar is arranged for making the second cut on the stalk of the plant above the first cut and below a lower boundary of the high protein gradient to separate the upper protein gradient from the lower protein gradient, the lower boundary being defined by a height along the stalk of the plant of the plant product closest to the ground surface that has a protein content meeting the threshold protein content.

In some example embodiments of the combine harvester of any previous or any combination of preceding example embodiments, the combine harvester further comprises a near-infrared spectroscopy (NIRS) mechanism in communication with the second header, and arranged to separately analyze a protein content of the high protein plant product harvested from the upper protein gradient of the plant and the protein content of the lower protein plant product harvested from the lower protein gradient of the plant.

In some example embodiments of the combine harvester of any previous or any combination of preceding example embodiments, the combine harvester further comprises an adjustment mechanism arranged to automatically adjust a height of the first header relative to the ground surface, and automatically adjust a height of a second header relative to the height of the first header and based on real-time feedback from the NIRS mechanism on the protein content of the high protein plant product harvested from the upper protein gradient of the plant and the protein content of the lower protein plant product harvested from the lower protein gradient of the plant so as to automatically adjust a height of the second cut.

In some example embodiments of the combine harvester of any previous or any combination of preceding example embodiments, the adjustment mechanism comprises a piston that, upon actuation, adjusts the height of the second header relative to the first header.

In some example embodiments of the combine harvester of any previous or any combination of preceding example embodiments, the adjustment mechanism is configured to: automatically adjust the height of the second header closer to the height of the first header in response to the real-time feedback from the NIRS mechanism that the lower protein plant product harvested from the lower protein gradient of the plant includes a percentage of plant product with a protein content that meets the threshold protein content for the upper protein gradient, or automatically adjust the height of the second header farther from the height of the first header in response to the real-time feedback from the NIRS mechanism that the higher protein plant product harvested from the upper protein gradient of the plant includes a percentage of plant product with a protein content that does not meet the threshold protein content for the upper protein gradient.

In some example embodiments of the combine harvester of any previous or any combination of preceding example embodiments, the separation mechanism comprises a partition arranged to divide the separation mechanism into a first separator in communication with the first header and a second separator in communication with the second header, and wherein a first conveyor of the first header is arranged to convey the lower protein plant product from the first header to the first separator to thresh and separate the lower protein plant product from the plant, and a second conveyor of the second header is arranged to convey the high protein plant product from the second header to the second separator to thresh and separate the high protein plant product from the plant.

In some example embodiments of the combine harvester of any previous or any combination of preceding example embodiments, the storage tank comprises a partition arranged to divide the storage tank into a first storage tank and a second storage tank, and wherein the lower protein plant product is stored in the first storage tank and the high protein plant product is stored in the second storage tank.

In some example embodiments of the combine harvester of any previous or any combination of preceding example embodiments, the plant is a soy plant producing soybeans and the threshold protein content for soybeans for the upper protein gradient of the soy plant is 40%, 42%, or 45% on a dry weight basis.

It will be appreciated that the above Summary is provided merely for purposes of summarizing some example aspects so as to provide a basic understanding of some aspects of the disclosure. As such, it will be appreciated that the above described example aspects are merely examples of some aspects and should not be construed to narrow the scope or spirit of the disclosure in any way. It will be appreciated that the scope of the disclosure encompasses many potential aspects, some of which will be further described below, in addition to those here summarized. Further, other features, aspects, and advantages of the disclosure will be apparent from a reading of the following detailed description taken in conjunction with the accompanying drawings which illustrate, by way of example, the principles of the described aspects.

### BRIEF DESCRIPTION OF THE FIGURES

In order to assist the understanding of aspects of the disclosure, reference will now be made to the appended drawings, which are not necessarily drawn to scale and in which like reference numerals refer to like elements. The drawings are exemplary only, and should not be construed as limiting the disclosure.

FIG. 1 illustrates a method flow diagram for a method of gradient harvesting plant product from a plant in a single pass using a combine harvester according to some embodiments of the present disclosure.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Seeds of soybean cultivars in the United States have an average composition of 40% protein in dry weights of cotyledons of ungerminated seeds. Hsu, et al. (1973). However, some soybean plants may be considered “high protein soybean plants” such that the population of high protein soybean plants or seeds has a greater frequency of high-protein soybeans than a control population of soybeans. In particular embodiments, the mean protein content (dry weight basis) of a population of soybeans harvested by the methods disclosed herein is greater than the mean protein content of a control population of soybeans. The control population of soybeans can be a population of commodity soybeans having a protein content of less than 40%, or between about 35% and about 40%, on a dry weight basis. As used herein, “control population” refers to a population of soybeans harvested from the whole plant, including both the upper section of soybeans and lower section of soybeans.

Notably, it has been found that a protein content gradient of soybean plants varies along a height of the soybean plant, such that the protein content of soybeans on a single plant may differ depending on what portion of the plant they grow, while the average protein composition of the soybeans of a plant remains about 40% by dry weight. For example, along a longitudinally-extending stalk of the soy plant, there may be a gradient of protein content that increases along the length of the stalk away from the ground surface. In this example, the soy plant may have an identified upper protein gradient including high protein plant product that meets a threshold protein content (e.g., 40% by dry weight) and a lower protein gradient including lower protein plant product with a protein content that is lower than the threshold protein content. In some variations of the foregoing embodiments, the soybeans having a high protein content comprise threshold protein content of at least about 40%, at least about 41%, at least about 42%, at least about 43%, at least about 44%, at least about 45%, at least about 46%, at least about 47%, at least about 48%, at least about 49%, at least about 50%, at least about 51%, or at least about 52% soy protein on a dry weight basis. In certain variations, the soybeans comprise at least about 42% soy protein on a dry weight basis. In some variations, the soybeans comprise at least about 45% soy protein on a dry weight basis. In other variations, the soybeans comprise at least about 48% soy protein on a dry weight basis. In specific embodiments, the threshold for the high protein plant product (e.g., soybean seeds) is a relative protein content when compared to the lower protein plant product. For example, the threshold can be a protein content that is greater than the lower protein plant product by at least about 0.25%, 0.5%, 0.75%, 1.0%, 1.5%, 2.0%, 2.5%, 3.0%, 3.5%, 4.0%, 4.5%, 5%, 6%, 7%, 8%, 9%, 10%, 12%, 14%, 16%, 18%, 20%, or more.

In some embodiments, the protein content of the soybeans having a high protein content may be described as average protein content of the soybeans. In certain embodiments, the protein content of the soybeans having a high protein content may be an average protein content of the soybeans for a given mass of soybeans (e.g., per 1 kilogram mass). In other embodiments, the protein content of the soybeans having a high protein content may be characterized by a distribution of protein contents. In other embodiments, the soybeans or population of soybeans have a threshold protein content. In some variations, the threshold protein content is at least about 40%, at least about 41%, at least about 42%, at least about 43%, at least about 44%, at least about 45%, at least about 46%, at least about 47%, at least about 48%, at least about 49%, at least about 50%, at least about 51%, or at least about 52% soy protein on a dry weight basis.

The percent composition of a given component in a soybean or sample of soybeans may be described on an “as-is” basis or on a “dry-weight” basis. The percent composition of a given component may be converted between an “as-is” basis and a “dry-weight” basis using the following equation:

(protein content,“dry-weight” basis,%)=(protein content,“as-is” basis,%)/((100%)−(moisture content,%)) Eq. 1

The word “protein” in the above equation may be interchanged with any other soybean component for which conversion between an “as-is” and a “dry-weight” basis is needed, including, e.g., oil. The moisture content of a soybean or soybean product may be determined using any suitable methods or techniques known in in the art. See e.g., Eys, J. E. Van. Manual of Quality Analyses For Soybean Products in The Feed Industry, 2nd ed. U.S. Soybean Export Council.

The soybeans comprising high protein content as employed by the methods of the present disclosure may be further characterized by one or more additional components present in the soybeans, such as oligosaccharide contents. The carbohydrate component of soybeans is comprised of three major oligosaccharides: sucrose, raffinose, and stachyose. Of the three, only sucrose is nutritionally useful and can be fully digested by monogastric animals. Raffinose and stachyose are considered anti-nutritional units because they cannot be digested due to the lack of a-galactosidase activity in the gut of monogastric animals.

In some embodiments, the soybeans having a high protein content may also comprise low raffinose and/or stachyose content. In some embodiments, the soybeans of the present disclosure comprise less than or equal to about 0.13%, less than or equal to about 0.11%, less than or equal to about 0.1%, less than or equal to about 0.07%, less than or equal to about 0.06%, less than or equal to about 0.03%, or less than or equal to about 0.01% raffinose on a dry weight basis. In other embodiments, the soybeans of the present disclosure comprise between about 0% to about 0.13% raffinose on a dry weight basis. In some embodiments, the soybeans of the present disclosure comprise about 0.01%, 0.03%, 1.06%, 0.07%, 0.10%, 0.11% and 0.13% raffinose on a dry weight basis, including all integers and fractions thereof. In some embodiments, the soybeans of the present disclosure comprise less than or equal to about 0.13%, less than or equal to about 0.11%, less than or equal to about 0.1%, less than or equal to about 0.07%, less than or equal to about 0.06%, less than or equal to about 0.03%, or less than or equal to about 0.01% stachyose on a dry weight basis. In other embodiments, the soybeans of the present disclosure comprise about 0.02%, 0.05%, 0.07%, 0.12%, 0.16%, 0.21%, 0.26%, 0.34%, 0.38%, 0.48%, 0.49%, 0.51%, 0.55%, 0.59%, 0.63%, 0.67%, 0.78%, 0.80%, 0.85%, 0.91%, 0.96%, 1.12%, 1.19%, 1.23%, 1.28%, 1.33%, 1.38%, 1.45%, 1.49%, 1.56%, 1.57%, 1.63%, 1.68%, 1.71%, 1.73%, 1.75% stachyose on a dry weight basis, including all integers and fractions thereof.

In some embodiments, the soybeans comprise a combined raffinose and stachyose content of between about 0.02% and 1.75%.

The present invention is not limited to whether the soybeans comprise transgenic polynucleotides or proteins. The soybeans used in the Examples herein are non-transgenic and there are circumstances when using soybeans lacking transgenic traits, genome edits, or any other form of mutation (i.e. a change in a polynucleotide sequence) is necessary and/or beneficial. However, combining the teachings herein with a wide range of transgenic plants, or plants containing genome edits or any other form of mutation to confer new traits or combinations thereof is also envisioned.

In some embodiments of the present disclosure, the soybeans having high protein content as utilized herein may be characterized by any suitable methods known in the art for genetic analysis. In some embodiments, the soybeans as utilized herein may be characterized by genetic analysis as comprising one or more genetic markers associated with high protein content and/or one or more genetic markers associated with low raffinose content and/or one or more genetic markers associated with low stachyose content as described above. In some embodiments, the soybeans as utilized herein may be characterized by genetic analysis as comprising one or more genetic markers associated with high protein content.

High protein content soy ingredients are desirable for a variety of food products and applications. In specific embodiments, the protein-enriched soy composition is incorporated into a food product, a beverage product, a dietary supplement product or other product. High protein soy ingredients are desirable for their nutritional properties, as well as the functional properties derived from their protein content. These functional properties are the intrinsic physicochemical characteristics which affect the behavior of a food ingredient in food systems during processing, manufacturing, storage and preparation. Such functional properties include water holding, oil binding, emulsification, foam capacity, gelation, whipping capacity, viscosity and others. Functional properties are important in determining the quality (nutritional, sensory, physicochemical and organoleptic properties) of the final product as well as facilitating processing such as improved machinability of cookie dough or slicing of processed meats. Therefore functional properties of food proteins are important in food processing and food product formulation. The functional behavior of proteins in food is influenced by some physicochemical properties of the proteins such as their size, shape, amino acid composition and sequence, net charge, charge distribution, hydrophobicity, hydrophilicity, type of structures, molecular flexibility/rigidity in response to external environment such as pH, temperature, salt concentration or interaction with other food constituents. Thus, the soybeans with higher protein content are generally more desirable.

In certain aspects, provided are also food and beverage products incorporating or produced using the protein-enriched soy compositions disclosed herein. Such protein-enriched soy compositions may be used for protein fortification in various food and beverage products, including for example, in juice based high acid beverages, allergen-free non-dairy low acid beverages, plant-based yogurts, plant-based ice-creams, bakery products, baked snacks, cream soups, meat analogs, and cheese analogs.

In some embodiments, suitable food products may include, for example, soups, sauces, salad dressings, hummus, breads, cookies, crackers, nutritional bars, meal replacement products, and snacks. In some variations, the food product incorporating or produced from the protein-enriched soy compositions herein is a bakery product.

Conventionally, the step of providing soybeans having a high protein content may be achieved by sorting and/or harvesting practices selective for soybeans having high protein content. For example, in some embodiments, a plurality of soybeans are separated according to protein content to provide at least a portion of soybeans having a protein content of at least 40%, 42%, or 45% on a dry weight basis, from the plurality of soybeans. This separation of the plurality of soybeans according to protein content may be a batch process, a continuous process, or a high-throughput process.

Other methods that are conventionally used for harvesting high protein content soybeans may comprise harvesting soybeans according to the height gradient on the soy plant, e.g., harvesting soybeans growing higher on a given soy plant (i.e., in a top portion of the plant). This may be accomplished manually or mechanically using a combine harvester that is able to adjust height of its header to account for the height gradient of the high protein soybeans on the soy plant. However, in this conventional method, although the desirable high protein soybeans in the top portion of the plant are harvested, the lower protein soybeans (i.e., in a bottom portion of the plant) are ignored or are harvested on a second pass through the field.

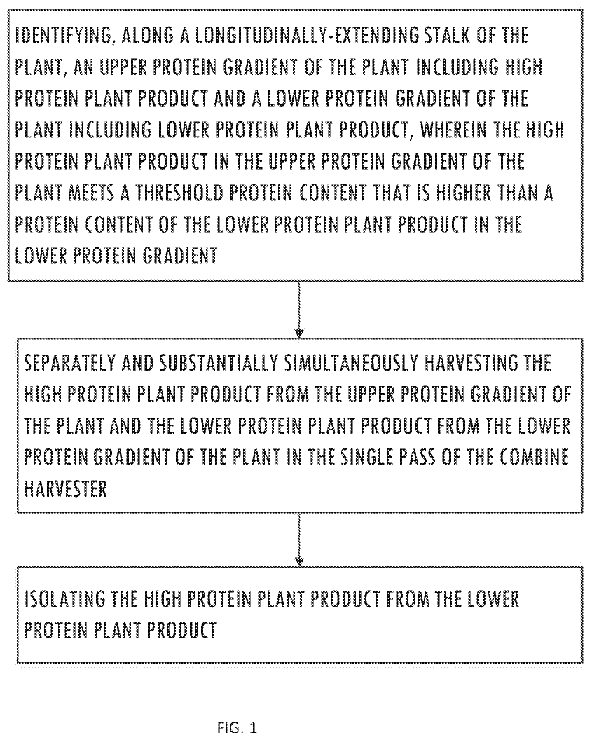

As illustrated in FIG. 1, the present disclosure improves on this conventional method by identifying, along a longitudinally-extending stalk of the plant, an upper protein gradient of the plant including high protein plant product and a lower protein gradient of the plant including lower protein plant product, wherein the high protein plant product in the upper protein gradient of the plant meets a threshold protein content that is higher than a protein content of the lower protein plant product in the lower protein gradient. In some example embodiments, the plant is a soy plant producing soybeans and the threshold protein content for soybeans for the upper protein gradient of the soy plant is 40%, 42%, or 45% on a dry weight basis. In specific embodiments, the high protein plant product (e.g., soybean seeds) have a protein content that is greater than the lower protein plant product by at least about 0.25%, 0.5%, 0.75%, 1.0%, 1.5%, 2.0%, 2.5%, 3.0%, 3.5%, 4.0%, 4.5%, 5%, 6%, 7%, 8%, 9%, 10%, 12%, 14%, 16%, 18%, 20%, or more. Once identified, then the high protein plant product from the upper protein gradient of the plant and the lower protein plant product from the lower protein gradient of the plant are separately and substantially simultaneously harvested in a single pass of a combine harvester, and the high protein plant product is isolated from the lower protein plant product.

Example embodiments of a combine harvester for use with this method include a combine harvester with two internal systems running in tandem and/or a combine harvester with a single internal system that alternates in harvesting the high protein plant product and the low protein plant product. Example embodiments include combines with two full headers (i.e., the first and second headers) stacked on top of one another in a vertical direction.

In some example embodiments, the combine harvester comprises a first header including a first cutter bar for making a first cut on a stalk of the plant proximate to a ground surface to detach the plant from the ground surface. A second header including a second cutter bar for making a second cut on the stalk of the plant above the first cut to separate an upper protein gradient of the plant from a lower protein gradient of the plant, the upper protein gradient including high protein plant product and the lower protein gradient of the plant including lower protein plant product, wherein the high protein plant product in the upper protein gradient of the plant meets a threshold protein content that is higher than a protein content of the lower protein plant product in the lower protein gradient. The first and second cutter bars may be independently controlled by a control unit of the combine harvester.

In some example embodiments, the second cutter bar is arranged for making the second cut on the stalk of the plant above the first cut and below a lower boundary of the high protein gradient to separate the upper protein gradient from the lower protein gradient, the lower boundary being defined by a height along the stalk of the plant of the plant product closest to the ground surface that has a protein content meeting the threshold protein content. In certain embodiments, the second cutter bar makes the first cut on the stalk of the plant below the lower boundary of the high protein gradient independent of when the first cutter bar makes a cut. In certain embodiments, the second cutter bar makes the first cut on the stalk of the plant below the lower boundary of the high protein gradient independent of where the first cutter bar makes a cut on the plant body. In certain embodiments, the second cutter bar makes the first cut on the stalk of the plant below the lower boundary of the high protein gradient and the first cutter bar makes no cut on the plant, optionally leaving the lower portion of the plant connected to and/or or embedded in the soil.

The second header may comprise a reel. A position of the reel may be adjusted to match crop conditions and may be in contact with the upper protein gradient of the plant. For example, the position of the reel may be adjusted based on where the lower boundary is defined along the stalk of the plant. In some embodiments, the first header also comprises a reel (not shown). Each of the first and second headers may also comprise respective augers. The first and second augers may separately collect the cut lower protein plant product and high protein plant product from the respective cutter bars and direct them to respective conveyors, which will convey them to the separation mechanism.

The combine harvester may also comprise a separation mechanism arranged to separately thresh and separate the lower protein plant product from the plant and thresh and separate the high protein plant product from the plant. In some example embodiments the separation mechanism comprises a partition arranged to divide the separation mechanism into a first separator in communication with the first header and a second separator in communication with the second header, and wherein a first conveyor of the first header is arranged to convey the lower protein plant product from the first header to the first separator to thresh and separate the lower protein plant product from the plant, and a second conveyor of the second header is arranged to convey the high protein plant product from the second header to the second separator to thresh and separate the high protein plant product from the plant. The partition may be used to retrofit a separation mechanism for an existing combine harvester or the combine harvester may be manufactured with two separators. For example, the partition may comprise a wall structure with a bearing to support the wall. However, the combine harvester may comprise a single separation mechanism that alternates between threshing and separating the lower protein plant product from the plant and the high protein plant product from the plant.

A storage tank arranged to isolate the high protein plant product from the lower protein plant product. The storage tank may comprise a partition arranged to divide the storage tank into a first storage tank and a second storage tank. The first storage tank may be in communication with the first separator and the second storage tank may be in communication with the second separator. The lower protein plant product may be stored in the first storage tank and the high protein plant product is stored in the second storage tank, or vice versa. The partition may be used to retrofit a storage tank on an existing combine harvester or the combine harvester may be manufactured with two storage tanks. However, the combine harvester may comprise a single storage tank that stores the high protein plant product, while an outlet in communication with the separating mechanism ejects the lower protein plant product from the combine harvester and back onto the ground surface or into a separate processing stream or alternate storage tank.

The storage tank may comprise one or more valves (e.g., one-way valves). For example, there may be a single valve associated with each storage tank, which can be independently controlled to allow for either the high protein plant product or the lower protein plant product to be offloaded. Optionally, the valves can be opened simultaneously to offload both the high protein plant product or the lower protein plant product at the same time to different trucks, storage facilities, etc. A conveyor arm may also be utilized instead of or in addition to the valve(s) so as to independently transport each of the high protein plant product or the lower protein plant product out of the combine harvester.

In some example embodiments, a near-infrared spectroscopy (NIRS) mechanism in communication with the second header is arranged to separately analyze a protein content of the high protein plant product harvested from the upper protein gradient of the plant and the protein content of the lower protein plant product harvested from the lower protein gradient of the plant. The NIRS mechanism may be onboard the combine harvester or in wired or wireless communication with the combine harvester through one or more known methods of communication. An overview of NIRS measurements is detailed in Zhu et al. Determination of soybean routine quality parameters using near-infrared spectroscopy. Food Sci. Nutr. 2018, 6, 1109-1118. Samples are placed in position relative to a light source and detector for an near-infrared spectrometer, such as a Fourier-transform near-infrared spectrometer (FT-NIR) with a scanning spectral range of 3700-15,000/cm. Spectra are collected over the range 4000-12,600/cm, at a resolution of 16/cm with 60 scan number, which containing the absorbance regions of the traits of interest (4000-9000/cm for protein, moisture, and fat). Each sample is scanned three times to eliminate differences caused by objective factors. The spectra are compared to calibration or standard spectra, the protein content of which has been confirmed by wet laboratory methods, such as the Kjeldahl method.

An overview of the Kjeldahl method of nitrogen determination is detailed in Part II. Sample preparation, working scale, instrumental finish and quality control. Crit. Rev. Anal. Chem. 2013; 43:224-272. First, the sample to be analyzed is heated to 360-410° C. and digested using concentrated sulfuric acid in the presence of a catalyst, such as selenium or copper. Next, the pH of the solution is raised using sodium hydroxide in order to convert any ammonium (NH4+) in solution (derived from nitrogen in the digested sample) in to ammonia gas (NH3), which is then distilled off into an aqueous HCl solution of known volume and concentration. This solution may then be titrated in order to determine its pH, and back-calculate the amount of nitrogen in the original sample. The approximate percent weight of protein in a sample is calculated by multiplying the percent weight of nitrogen in the original sample by a factor of 6.25.

The combine harvester may further comprise an adjustment mechanism arranged to automatically adjust a height of the first header relative to the ground surface, and automatically adjust a height of the second header relative to the height of the first header and based on real-time feedback from the NIRS mechanism. The real-time feedback from the NIRS mechanism may comprise data regarding the protein content of the high protein plant product harvested from the upper protein gradient of the plant and the protein content of the lower protein plant product harvested from the lower protein gradient of the plant so as to automatically adjust a height of the second cut. Data from the NIRS mechanism may be transmitted to the adjustment mechanism, which may automatically and continuously adjust the height of the respective headers based on the data received. The data may be transmitted to the adjustment mechanism using any one of known data communication methods.

The adjustment mechanism may be configured to automatically adjust the height of the second header closer to the height of the first header in response to the real-time feedback from the NIRS mechanism that the lower protein plant product harvested from the lower protein gradient of the plant includes a percentage of plant product with a protein content that meets the threshold protein content for the upper protein gradient. The percentage of plant product with the protein content that meets the threshold protein content may be based on an identified maximum percentage, i.e., the percentage is greater than 5%, greater than 10%, greater than 15% or the like. Alternatively, the adjustment mechanism may be configured to automatically adjust the height of the second header farther from the height of the first header in response to the real-time feedback from the NIRS mechanism that the higher protein plant product harvested from the upper protein gradient of the plant includes a percentage of plant product with a protein content that does not meet the threshold protein content for the upper protein gradient. The percentage of plant product with the protein content that does not meet the threshold protein content may be based on an identified maximum percentage, i.e., the percentage is greater than 5%, greater than 10%, greater than 15% or the like.

In certain embodiments the adjustment mechanism comprises a piston that, upon actuation, adjusts the height of the second header relative to the first header. The piston may be tied to an existing hydraulics system in the combine harvester, such that the hydraulic system is retrofit to actuate the piston(s). However, any other type of adjustment mechanism may be used, such as, for example, a retracting arm, a winch, a magnet, and the like.

The combine harvester disclosed herein may also comprise a fan and/or sifter. A single fan's airflow can be diverted into two identical sifting systems. The combine harvesters as disclosed herein may also comprise any and all components typical to a combine harvester that may be integrated with the described elements. Additionally, while soybean plants are discussed in reference to the present method and related combine harvester, other plants may also be gradient harvested as contemplated. For example, and without limitation, yellow pea and other crops exhibiting a yield gradient across the plant body may be similarly gradient harvested using the combine harvesters contemplated herein.

Many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed herein and that modifications and other embodiments are intended to be included within the scope of the appended claims. Although specific terms are employed herein, they are used in a generic and descriptive sense only and not for purposes of limitation.

What is claimed is:

1. A method of gradient harvesting plant product from a plant in a single pass using a combine harvester, the plant having a protein content gradient that varies along a height of the plant, the method comprising:
identifying, along a longitudinally-extending stalk of the plant, an upper protein gradient of the plant including high protein plant product and a lower protein gradient of the plant including lower protein plant product, wherein the high protein plant product in the upper protein gradient of the plant meets a threshold protein content that is higher than a protein content of the lower protein plant product in the lower protein gradient;
separately and substantially simultaneously harvesting the high protein plant product from the upper protein gradient of the plant and the lower protein plant product from the lower protein gradient of the plant in the single pass of the combine harvester; and
isolating the high protein plant product from the lower protein plant product.

2. The method of claim 1, wherein identifying the upper protein gradient and the lower protein gradient comprises identifying a lower boundary of the high protein gradient, the lower boundary being defined by a height along the stalk of the plant of the plant product closest to the ground surface that has a protein content meeting the threshold protein content of the upper protein gradient.

3. The method of claim 2, wherein the combine harvester comprises a first header including a first cutter bar and a second header including a second cutter bar, and wherein separately and substantially harvesting the high protein plant product from the upper protein gradient of the plant and the lower protein plant product from the lower protein gradient of the plant in the single pass comprises:
making, using the first cutter bar, a first cut on the stalk of the plant proximate to the ground surface to detach the plant from the ground surface; and
substantially simultaneously making, using the second cutter bar, a second cut on the stalk of the plant above the first cut and below the lower boundary to separate the upper protein gradient from the lower protein gradient.

4. The method of claim 3, further comprising separately analyzing, using a near-infrared spectroscopy (NIRS) mechanism in communication with the second header, a protein content of the high protein plant product harvested from the upper protein gradient of the plant and a protein content of the lower protein plant product harvested from the lower protein gradient of the plant.

5. The method of claim 4, wherein separately and substantially harvesting the high protein plant product from the upper protein gradient of the plant and the lower protein plant product from the lower protein gradient of the plant further comprises:
automatically adjusting a height of the first header relative to the ground surface; and
automatically adjusting a height of the second header relative to the first header and based on real-time feedback from the NIRS mechanism on the protein content of the high protein plant product harvested from the upper protein gradient of the plant and the protein content of the lower protein plant product harvested from the lower protein gradient of the plant so as to automatically adjust a height of the second cut.

6. The method of claim 5, wherein automatically adjusting the height of the second header comprises:
automatically adjusting the height of the second header closer to the height of the first header in response to the real-time feedback from the NIRS mechanism that the lower protein plant product harvested from the lower protein gradient of the plant includes a percentage of plant product with a protein content that meets the threshold protein content for the upper protein gradient, or
automatically adjusting the height of the second header farther from the height of the first header in response to the real-time feedback from the NIRS mechanism that the higher protein plant product harvested from the upper protein gradient of the plant includes a percentage of plant product with a protein content that does not meet the threshold protein content for the upper protein gradient.

7. The method of claim 3, wherein the combine harvester comprises a separation mechanism, and wherein separately and substantially simultaneously harvesting the high protein plant product from the upper protein gradient of the plant and the lower protein plant product from the lower protein gradient of the plant comprises separately threshing and separating the lower protein plant product from the plant and threshing and separating the high protein plant product from the plant.

8. The method of claim 7, wherein the separation mechanism comprises a partition arranged to divide the separation mechanism into a first separator in communication with the first header and a second separator in communication with the second header, and wherein separately and substantially simultaneously harvesting the high protein plant product from the upper protein gradient of the plant and the lower protein plant product from the lower protein gradient of the plant comprises conveying, using a first conveyor, the lower protein plant product from the first header to the first separator to thresh and separate the lower protein plant product from the plant, and conveying, using a second conveyor, the high protein plant product from the second header to the second separator to thresh and separate the high protein plant product from the plant.

9. The method of claim 1, wherein the combine harvester comprises a storage tank, and wherein isolating the high protein plant product from the lower protein plant product comprises isolating the high protein plant product from the lower protein plant product in the storage tank.

10. The method of claim 1, wherein the plant is a soy plant producing soybeans and the threshold protein content for the upper protein gradient of the soy plant is 40%, 42%, or 45% on a dry weight basis.

11. The method of claim 1, wherein the high protein plant product is used to create a protein-enriched soy protein composition, the method comprising:
subjecting the isolated high protein plant product to one of defatting by solvent extraction, desolventizing, and contacting with a polar solvent, to arrive at a protein-enriched soy protein composition that comprises at least about 75% soy protein on a dry weight basis.

12. The method of claim 11 wherein the protein-enriched soy protein composition that comprises at east about 75% soy protein on a dry weight basis is a soy protein concentrate (SPC).

13. A method of producing a food product, a beverage product, a dietary supplement product or other product, comprising the protein-enriched soy composition according to claim 12.

14. A combine harvester for gradient harvesting plant product from a plant in a single pass, the plant having a protein content gradient that varies along a height of the plant, the combine harvester comprising:
a first header including a first cutter bar for making a first cut on a stalk of the plant proximate to a ground surface to detach the plant from the ground surface;
a second header including a second cutter bar for making a second cut on the stalk of the plant above the first cut to separate an upper protein gradient of the plant from a lower protein gradient of the plant, the upper protein gradient including high protein plant product and the lower protein gradient of the plant including lower protein plant product, wherein the high protein plant product in the upper protein gradient of the plant meets a threshold protein content that is higher than a protein content of the lower protein plant product in the lower protein gradient;
a separation mechanism arranged to separately thresh and separate the lower protein plant product from the plant and thresh and separate the high protein plant product from the plant; and
a storage tank arranged to isolate the high protein plant product from the lower protein plant product.

15. The combine harvester of claim 14, wherein the second cutter bar is arranged for making the second cut on the stalk of the plant above the first cut and below a lower boundary of the high protein gradient to separate the upper protein gradient from the lower protein gradient, the lower boundary being defined by a height along the stalk of the plant of the plant product closest to the ground surface that has a protein content meeting the threshold protein content.

16. The combine harvester of claim 14, further comprising a near-infrared spectroscopy (NIRS) mechanism in communication with the second header, and arranged to separately analyze a protein content of the high protein plant product harvested from the upper protein gradient of the plant and the protein content of the lower protein plant product harvested from the lower protein gradient of the plant.

17. The combine harvester of claim 15, further comprising an adjustment mechanism arranged to automatically adjust a height of the first header relative to the ground surface, and automatically adjust a height of a second header relative to the height of the first header and based on real-time feedback from the NIRS mechanism on the protein content of the high protein plant product harvested from the upper protein gradient of the plant and the protein content of the lower protein plant product harvested from the lower protein gradient of the plant so as to automatically adjust a height of the second cut.

18. The combine harvester of claim 17, wherein the adjustment mechanism is configured to:
automatically adjust the height of the second header closer to the height of the first header in response to the real-time feedback from the NIRS mechanism that the lower protein plant product harvested from the lower protein gradient of the plant includes a percentage of plant product with a protein content that meets the threshold protein content for the upper protein gradient, or
automatically adjust the height of the second header farther from the height of the first header in response to the real-time feedback from the NIRS mechanism that the higher protein plant product harvested from the upper protein gradient of the plant includes a percentage of plant product with a protein content that does not meet the threshold protein content for the upper protein gradient.

19. The combine harvester of claim 15, wherein the separation mechanism comprises a partition arranged to divide the separation mechanism into a first separator in communication with the first header and a second separator in communication with the second header, and wherein a first conveyor of the first header is arranged to convey the lower protein plant product from the first header to the first separator to thresh and separate the lower protein plant product from the plant, and a second conveyor of the second header is arranged to convey the high protein plant product from the second header to the second separator to thresh and separate the high protein plant product from the plant.

20. The combine harvester of claim 15, wherein the plant is a soy plant producing soybeans and the threshold protein content for soybeans for the upper protein gradient of the soy plant is 40%, 42%, or 45% on a dry weight basis.
